(19) United States
(12) Patent Application Publication
(10) Pub. No.: US 2022/0072211 A1
(43) Pub. Date: Mar. 10, 2022

(54) HEMOFILTRATION DEVICE, SYSTEM AND METHOD FOR A HIGH BLOOD FLOW EXTRACORPOREAL CIRCUIT

(71) Applicant: UNIVERSITY OF MARYLAND, BALTIMORE, Baltimore, MD (US)

(72) Inventors: Alison GRAZIOLI, Laurel, MD (US); Joseph RABIN, Silver Spring, MD (US); Ronson MADATHIL, Baltimore, MD (US); Rashmikant SHAH, Farmington Hills, MI (US)

(21) Appl. No.: 17/415,841

(22) PCT Filed: Dec. 20, 2019
(86) PCT No.: PCT/US19/67740

§ 371 Date: Jun. 18, 2021

Related U.S. Application Data
(60) Provisional application No. 62/782,429, filed on Dec. 20, 2018.

(51) Int. Cl. A61M 1/36; A61M 1/16; A61M 1/34

(52) U.S. Cl. CPC A61M 1/367; A61M 1/3479; A61M 1/1672; A61M 1/1619

(57) ABSTRACT

Disclosed is a hemofiltration device, system and method for rapid solute removal from a patient's blood. The device, and method employ a hemofiltration assembly for a high blood flow extracorporeal circuit, such as an ECMO circuit, configured to achieve high-efficiency, high-flux convective solute clearance, and optionally diffusive solute clearance, and include one or more hemofilters having greater filter medium surface area in a circuit having greater flow rates than previously implemented RRT modalities, and may offer rapid clearance of toxins, including those not currently dialyzable (e.g., those with high volumes of distribution).

### CROSS REFERENCE TO RELATED APPLICATION

This application claims the benefit of U.S. Provisional Application Ser. No. 62/782,429 titled “Hemofiltration Device for a High Blood Flow Extracorporeal Circuit,” filed Dec. 20, 2018 by the inventors herein, which application is incorporated herein by reference in its entirety.

### FIELD OF THE INVENTION

The present invention relates generally to devices and methods for managing blood flow in extracorporeal circuits, and more particularly to a device and method for rapid solute removal from a patient's blood using an extracorporeal membrane oxygenation circuit equipped with in-line, high-efficiency and high-flux hemofiltration.

### BACKGROUND

Solute clearance from a patient's blood is typically achieved through use of a Renal Replacement Therapy (“RRT”), which can either occur through conventional intermittent hemodialysis or continuous renal replacement therapy (“CRRT”). In hemodynamically unstable patients, CRRT can be achieved via a circuit placed at a dedicated access site or in-line with an ECMO apparatus. Using CRRT to achieve convective clearance has two significant limitations.

First, convective clearance is determined by the volume of hemofiltration over time which is limited by the system's blood flow, with higher blood flows allowing for greater clearance. Blood flow rates in CRRT circuits do not typically exceed 300 mL/minute due to smaller diameter access catheters (e.g., 12-14 French) and tubing. While higher hemofiltration rates alone may generate enhanced solute removal by increasing solute drag across the hemofilter membranes, currently achievable hemofiltration rates are restricted by filtration fraction (hemofiltration rate/blood flow in the circuit) with higher filtration fractions of >20%-30%, leading to excessive hemoconcentration and, by proxy, stagnation and risk of clotting. Hemoconcentration, in turn, is a function of blood flow, which in conventional CRRT circuits is limited to 150-300 ml/min due to smaller diameter access catheters (e.g., 12-14 French) and tubing. Both the impediments of filtration fraction ceiling and lower blood flow can be addressed with the use of systemic anticoagulation and large-diameter catheters, respectively.

A second limitation of conventional renal replacement therapy (“RRT”) (continuous and intermittent modalities) is the use of a single, standard-size hemofilter or hemodialyzer. These membranes are designed to accommodate blood flow ranges attainable through dialysis catheters (up to 400 ml/minute) or via cannulation of peripheral arteriovenous access sites, which can achieve up to a maximum of about 600 ml/minute.

Thus, the ability of current renal replacement therapy modalities to achieve rapid solute removal is limited by both membrane surface area and blood flow rate; therefore, there remains a need in the art for new devices and methods capable of achieving rapid solute removal from a patient's blood that will accommodate higher blood flows and use membranes capable of handling higher blood volumes while maintaining hemodynamic stability.

### SUMMARY OF THE INVENTION

Disclosed herein is a hemofiltration device, system and method for rapid solute removal from a patient's blood. Because the efficiency of convective solute removal is dependent upon both the hemofilter surface area and rate of hemofiltration, proportionally increasing the hemofilter capacity to accommodate 5-7 L/minute achieved by an extracorporeal circuit, such as an extracorporeal membrane oxygenation (“ECMO”) circuit, could dramatically enhance the efficiency of solute clearance over previously known devices and methods, such as 10-15 times higher, and possibly even 30 times higher, than that of traditional CRRT, and 6 times that of hemodialysis.

The device and system described herein employ a hemofiltration assembly for a high blood flow extracorporeal circuit configured to achieve high-efficiency, high-flux convective solute clearance, and optionally diffusive solute clearance, and include one or more hemofilters having greater filter medium surface area in a circuit having greater flow rates than previously implemented RRT modalities. Performing hemofiltration pursuant to the device, system, and methods set forth herein at high blood flow rates with high-flux membranes offers highly efficient solute clearance well beyond current RRT capabilities. Traditional RRT limitations of blood flow dependency and filtration fraction can be overcome to offer rapid clearance of toxins, including those not currently dialyzable (e.g., those with high volumes of distribution).

In accordance with certain aspects of an embodiment of the invention, a device for processing blood flow in an extracorporeal circuit is provided, comprising: a high-flux hemofilter assembly, the high-flux hemofilter assembly further comprising one or more blood inlets in fluid communication with a patient blood drainage line; one or more blood outlets in fluid communication with a patient blood return line; and an effluent outlet; wherein the high-flux hemofilter assembly is configured to provide solute clearance on blood supplied through the one or more blood inlets at a blood flow rate of 1-7 L/min, and wherein the solute clearance is effective to cause small solute clearance (e.g., creatinine or urea) from the blood supplied through the one or more blood inlets at a rate of 24-90 L/hr.

In accordance with further aspects of an embodiment of the invention, a system for processing blood flow in an extracorporeal circuit is provided, comprising: a high-flux hemofilter assembly, the high-flux hemofilter assembly further comprising one or more blood inlets in fluid communication with a patient blood drainage line; one or more blood outlets in fluid communication with a patient blood return line; and an effluent outlet; wherein the high-flux hemofilter assembly is configured to provide solute clearance on blood supplied through the one or more blood inlets at a blood flow rate of 1-7 L/min, and wherein the solute clearance is effective to cause small solute clearance (e.g., creatinine or urea) from the blood supplied through the one or more blood inlets at a rate of 24-90 L/hr; and a fluid selected from the group consisting of a replacement fluid and a dialysate, wherein the fluid is in fluid communication with the extracorporeal circuit. The same setup could also be used for diffusive clearance, whereby 1-7 L of blood flow would run countercurrent to dialysate flow (a range of 1-9 L/hr depending upon clearance goals).

In accordance with still further aspects of an embodiment of the invention, a method for processing blood flow in an extracorporeal circuit is provided, comprising the steps of:

providing a high-flux hemofilter assembly, the high-flux hemofilter assembly further comprising one or more blood inlets in fluid communication with a patient blood drainage line, one or more blood outlets in fluid communication with a patient blood return line, and an effluent outlet, wherein the high-flux hemofilter assembly is configured to provide solute clearance on blood supplied through the one or more blood inlets at a blood flow rate of 1-7 L/min, and wherein the solute clearance is effective to cause small solute clearance (e.g., creatinine or urea) from the blood supplied through the one or more blood inlets at a rate of 24-90 L/hr; providing a fluid selected from the group consisting of a replacement fluid and a dialysate, wherein said fluid is in fluid communication with the extracorporeal circuit; supplying blood from a patient to the high-flux hemofilter assembly at a blood flow rate of 1-7 L/min; and operating the high-flux hemofilter assembly to perform solute clearance on the blood effective to cause small solute clearance (e.g., creatinine or urea) from the blood at a rate of 24-90 L/hr.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is a schematic view of a system for a high blood flow extracorporeal circuit in accordance with certain aspects of an embodiment of the invention.

FIG. 2 is a schematic view of a system for a high blood flow extracorporeal circuit in accordance with further aspects of an embodiment of the invention.

FIG. 3 is a schematic view of a system for a high blood flow extracorporeal circuit in accordance with still further aspects of an embodiment of the invention.

FIG. 4 is photograph showing two hemofilters placed in parallel in-line with an ECMO circuit with effluent tubing to allow for gravity drainage in accordance with certain aspects of an embodiment of the invention.

FIG. 5 is a chart comparison of theoretical and achieved Cr clearance using 1 and 2 Sorin-14 hemofilters in-line with a high blood flow extracorporeal circuit.

FIG. 6 is a chart depicting a theoretical comparison between CVVH, conventional hemodialysis and extracorporeal circuit hemofiltration looking at small molecule clearance at various blood flows and different filtration fractions.

### DETAILED DESCRIPTION OF THE PREFERRED EMBODIMENTS

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

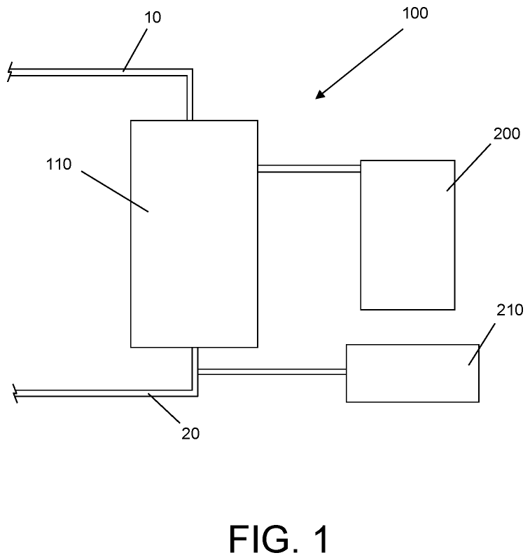

FIG. 1 is a schematic view of a high-efficiency, high-flux system 100 for solute clearance. Blood is directed from a patient via patient drainage line 10 to a high-flux hemofilter assembly 110 positioned in-line in, for example, an extracorporeal circuit, at a blood flow rate of preferably 1-7 L/min, and more preferably 4-5 L/min, where it undergoes solute clearance. Effluent waste produced during such solute clearance is collected at effluent waste 200. As detailed below, high-flux hemofilter assembly 110 is configured to effect small solute (e.g., creatinine or urea) clearance from the patient's blood of 24-90 L/hr at that flow rate, after which it is returned via line 20 to the patient. As further detailed below, replacement fluid 210, which in certain embodiments may include urea, is supplied to return line 20 to replenish nutrients that were lost during the solute clearance process. Replacement fluid 210 may be configured to supply replacement fluid to the blood pre- its delivery to high-flux hemofilter 110 or post-delivery. Alternatively, replacement fluid 210 may be configured to deliver replacement fluid (or dialysate) on the opposite side of the blood and across the membranes in high-flux hemofilter 110 in a direction counter current to the blood flow to achieve diffusive clearance.

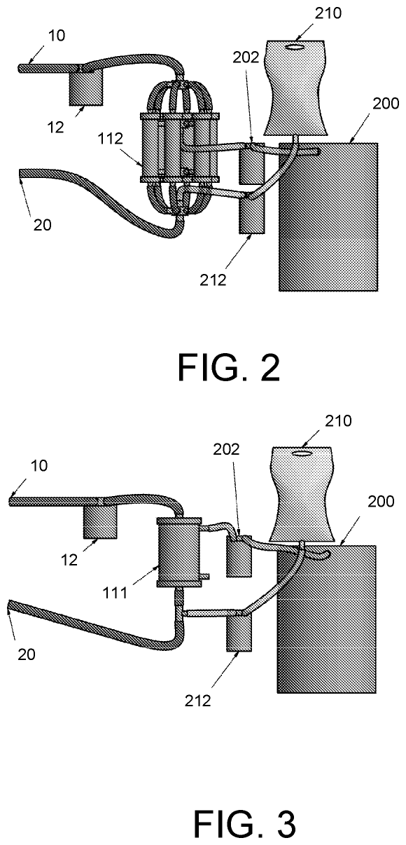

In an exemplary configuration, and with reference to FIG. 2, high-flux hemofilter assembly 110 comprises a plurality of high-flux hemofilters 112 arranged in the high blood flow circuit, such as an ECMO circuit, in parallel with one another. In such configuration, patient drainage line 10 preferably pulls blood from the patient via a high blood flow centrifugal pump 12. Upstream from parallel hemofilters 112, patient drainage line 10 separates into separate inlet lines that attach to respective inlets of each one of parallel hemofilters 112. Control and flow sensors (not shown) may be provided to ensure uniform blood flow through each of the separate hemofilters 112. Likewise, separate outlet branches from the blood outlet of each one of parallel hemofilters 112 come together to supply filtered blood to return line 20 for delivery back to the patient. Effluent removal from parallel hemofilters 112 is preferably supported by an effluent pump 202, which collects effluent waste in effluent waste container 200. In exemplary configurations, effluent pump 202 may operate to maintain an effluent flow rate of up to 2 L/min. Further, replacement fluid may be delivered to patient return line 20 via a replacement fluid pump 212. Replacement fluid pump 212 is preferably configured to match the hemofiltration rate unless additional fluid removal is desired. In some configurations, replacement fluid 210 may alternatively be supplied upstream from parallel hemofilters 112.

The configuration of FIG. 2 is modular in nature, such that a single one of parallel hemofilters 112 may readily be removed and replaced if and as necessary to maintain the intended operation. Likewise, such system is operated to ensure that blood flow through each one of parallel hemofilters 112 maintains a uniform pressure inside of each such parallel hemofilter 112, and uniform outflow from each one of parallel hemofilters 112. In particularly preferred embodiments, high-flux hemofilter assembly 110 may comprise 6-12 hemofilters placed in parallel in the extracorporeal circuit, and more preferably 6-10 filters, operating at a blood flow rate of preferably 4-6 L/min.

In the exemplary configuration of FIG. 2, parallel hemofilters 112 are readily commercially available hemofilters, and may comprise (by way of non-limiting example) in-line Sorin-14 (polyethersulfone membrane) hemoconcentrators, which are readily commercially available from Livallova USA, Inc. in Arvada, Colo., USA.

In another exemplary configuration, and with reference to FIG. 3, high-flux hemofilter assembly 110 comprises a single, larger hemofilter 111 placed in series with the high blood flow circuit, such as an ECMO circuit, and configured to maintain the ratios of blood flow to surface area that are achieved with traditional RRT modalities. In certain configurations, single, larger hemofilter 111 may have a filter membrane total surface area of 3-21 m². In this configuration, patient drainage line 10 preferably pulls blood from the patient via a high blood flow centrifugal pump 12, and supplies the patient's blood to a single inlet of hemofilter 111. Control and flow sensors (not shown) again may be provided to ensure uniform blood flow through hemofilter 111. Likewise, an outlet of hemofilter 111 supplies filtered blood to return line 20 for delivery back to the patient. Effluent removal from hemofilter 111 is preferably supported by an effluent pump 202, which collects effluent waste in effluent waste container 200. In exemplary configurations, effluent pump 202 may operate to maintain an effluent flow rate of up to 2 L/min. Further, replacement fluid 210 may be delivered to patient return line 20 via a replacement fluid pump 212. Replacement fluid pump 212 is preferably configured to match the hemofiltration rate unless additional fluid removal is desired. In some configurations, replacement fluid 210 may alternatively be supplied upstream from hemofilter 111.

In certain configurations, the system 100 may also include a flow diverter (not shown) positioned and configured to allow some portion of blood that is being delivered to high-flux hemofilter assembly 110 to bypass the filtration circuit within hemofilter assembly 110, such as by way of a flow diverter positioned upstream of hemofilter assembly 110, or even within hemofilter assembly 110. For particularly high-flow arrangements, it may be desirable to divert a portion of the blood flow to ensure adequate, continuous resupply to the patient.

Maintenance of blood flow to surface area ratios in such configurations is important to preserve the transmembrane pressures and solute sieving properties that standard hemofilter membranes are designed to sustain. In addition, this would prevent complications of hemolysis secondary to increased shear forces created by blood membrane interactions at higher pressures. Pressure monitors may be incorporated to allow for monitoring of transmembrane pressures and circuit pressures (not shown).

The use of hemofilters as described here and incorporated into a high-flow circuit can achieve solute clearance rates that exceed current RRT capabilities. For example, configuring a high-flow circuit with two in-line hemofilters placed in parallel in accordance with the foregoing description achieved creatinine clearance of between 11 and 12 L/hour. While traditional RRT provides adequate support for renal failure, its limited efficiency can prevent its application to other critical clinical scenarios. Patients suffering from toxin ingestion, such as drug overdose, endogenous toxic molecule production such as myoglobin in rhabdomyolysis, or cytokine release as occurs in sepsis, may require higher levels of solute clearance for potential therapeutic benefit. Thus, the high efficiency, high-flux clearance that can be achieved by the device, system and methods described herein may broaden the utilization of convective (or diffusive) solute removal as a possible therapeutic tool for a variety of disease states mediated by endogenous or exogenous toxins.

A potential limitation of highly efficient solute clearance includes the potential removal of beneficial drugs, electrolytes (e.g., phosphate), and important molecules such as micronutrients (e.g., B-6, B-12, ascorbic acid, etc.). Such removal of beneficial elements should be considered in conjunction with drug dosing modification and the supply of replacement fluid to include physiologic concentrations of important molecules at risk for depletion. Abrupt change in serum osmolality due to highly efficient solute clearance is another potential risk. This could be managed with the addition of urea or an alternative osmole to the replacement fluid 210 to avoid development of hemodynamic instability, cerebral edema, and dialysis disequilibrium.

In certain exemplary configurations, replacement fluid 210 (and/or dialysate) may be provided that is particularly configured to avoid the negative conditions discussed above. Such replacement fluid/dialysate 210 may include normal serum concentrations of removed substances. For example, in adult patients, substances that may be included in replacement fluid 210 or dialysate can include:

Phosphorous: 2.5-4.5 mg/dL, and most preferably 3.5 mg/dL

Iron: 60-170 m/dL, and most preferably 100 m/dL

Thiamine: 2.5-7.5 μg/dL, and most preferably 5 μg/dL

Riboflavin: 4-25 μg/dL, and most preferably 15 m/dL

Vitamin B-6: 5-50 m/dL, and most preferably 25 μg/dL

Vitamin B-12: 200-900 ng/ml, and most preferably 500 ng/ml

Niacin: 0.5-8.45 μg/mL, and most preferably 2 μg/mL

Folic Acid: 2.7-17.0 ng/mL, and most preferably 10 ng/mL

Vitamin C (ascorbic acid): 0.4-2.0 mg/dL, and most preferably 0.6 mg/dL Generally, the concentrations of the foregoing substances approximate normal serum concentrations.

Additionally, high-efficiency, high-flux solute clearance as described herein may cause rapid clearance of osmoles that could rapidly decrease a patient's serum osmolarity. Rapid decline in serum osmolarity (including rapid urea clearance) may cause neurologic symptoms (e.g., cerebral edema and dialysis equilibrium syndrome). Thus, system 100 may be configured to allow the clinician to maintain the patient's osmolarity in a range that is safe for the patient, and keep it from falling too rapidly by replacing or adding dialysis fluids with urea in varying concentrations. Urea may be incorporated in replacement fluid 210 (or dialysate) to a final concentration that could be chosen by the clinician to most closely match the patient's baseline to avoid rapid decline. For example, ranges of urea concentration in engineered replacement fluid or dialysate may range from 20-200 mg/dL, and more preferably 30-100 mg/dL. The addition of urea to replacement fluid 210 (or dialysate) could also be used for conventional renal replacement modalities where rapid urea clearance could cause harm. For example, initiation of a patient on intermittent hemodialysis for the first time (usually with higher baseline urea levels of 100 mg/dL or more) often requires stepwise lowering of urea in graduated levels over the span of three days to prevent dialysis disequilibrium syndrome. Use of engineered solutions tailored to the patient could obviate this need. This would allow for more rapid clearance and fewer treatment sessions without risking large osmolar shifts.

The risk of poor volumetric control leading to hemodynamic instability resulting from highly efficient solute clearance may also be addressed by addition of a volumetric infusion pump.

### EXAMPLE

A 62 year old 62 kg man with a history of hypertension, diabetes, coronary artery disease, and chronic kidney disease presented to a hospital with dyspnea. Left heart catheterization (LHC) was performed showing 99% occlusion of the right coronary artery. The patient was later transferred to another institution for additional percutaneous coronary intervention. Renal replacement therapy (RRT) was initiated at the outside hospital for non-oliguric acute kidney injury (AKI). During LHC on day of arrival, the patient suffered cardiac arrest and was placed on veno-arterial extracorporeal membrane oxygenation (ECMO) peri-arrest. The patient's AKI worsened and he developed oliguria within 24 hours.

After emergent initiation of ECMO, consent was obtained for continuation and for initiation of RRT (convective clearance for solute removal). Initially, fluid overload was managed using an in-line hemofilter as is already the standard of care in most ECMO units in the United States. The hemofilter, an in-line Sorin-14 (polyethersulfone membrane) hemoconcentrator (Livallova USA, Inc., Arvada, Colo., USA), had a blood flow throughput of ˜1 L/minute per flow meter measurement and was paced before the oxygenator, with proper technique and with close monitoring for safety purpose. The Sorin-14 hemofilter has characteristics that, at minimum, match high-flux hemodialyzers with a sieving cut-off of 65,000 Da and is well suited for high-pressure convective solute clearance. To establish the validity of baseline convective qualities, effluent electrolyte concentrations from both the Sorin-14 and the PRISMAFLEX M-150 set AN 69 (Baxter International, Deerfeild, Ill., USA) conventional continuous renal replacement therapy (CRRT) membrane were concurrently compared in another patient and were nearly identical (data not shown). During the patient's first solute clearance treatment period (treatment one), 5 L of PrismaSATE replacement fluid (Baxter International) was administered and 5 L of hemofiltration was performed using the hemofilter. This was achieved by first delivering 500 cc increments of replacement fluid via the ECMO circuit followed by the exact physician-matched removal of the same effluent volume (500 cc) via the hemofilter using gravity drainage. This process was repeated over the span of 50 minutes until the total effluent removed and replacement fluid volume administered each totaled 5 L. Risk of volume depletion was minimized by exactly matching the amount of replacement fluid given to the amount of effluent removed. Hemodynamics remained stable with mean arterial pressure (MAP) range of 80-90.

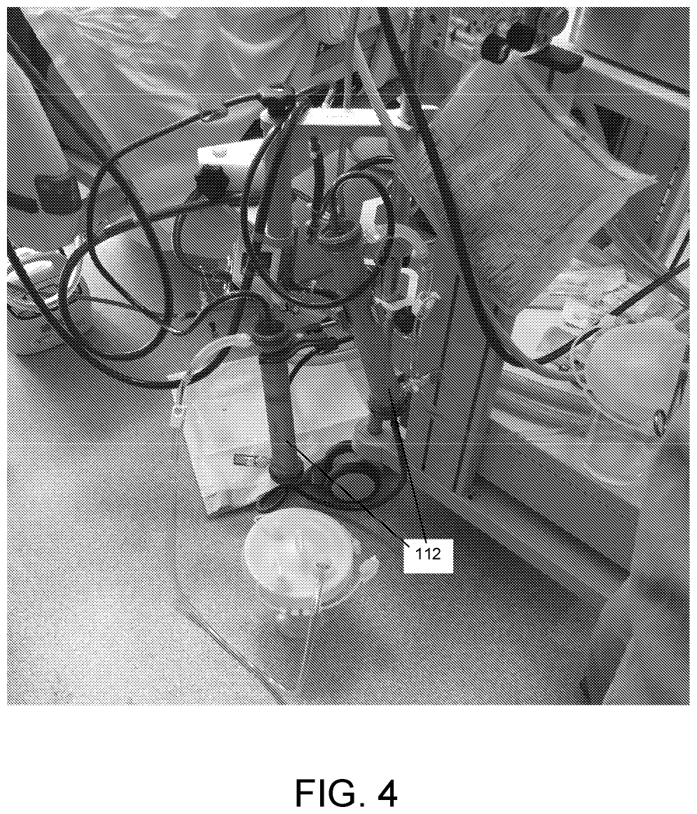

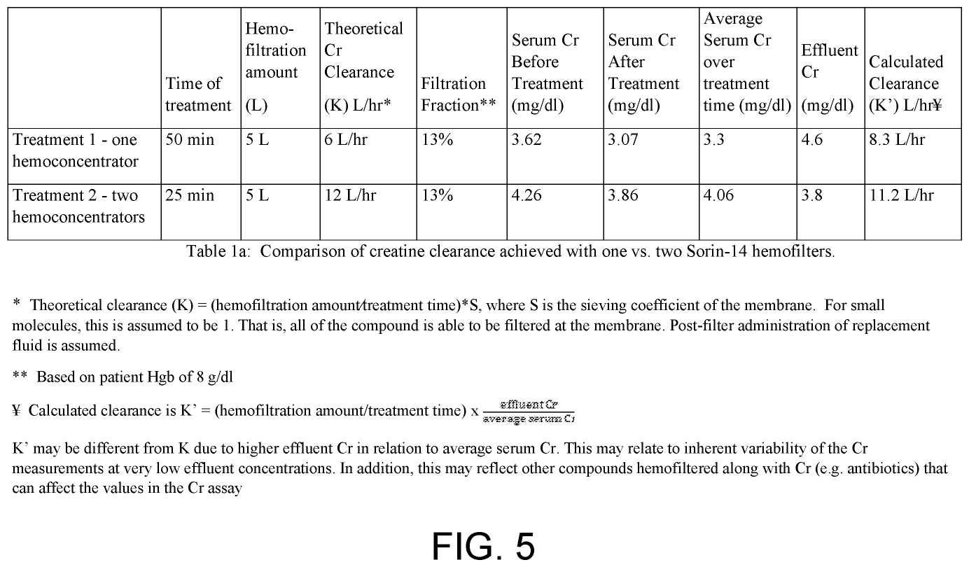

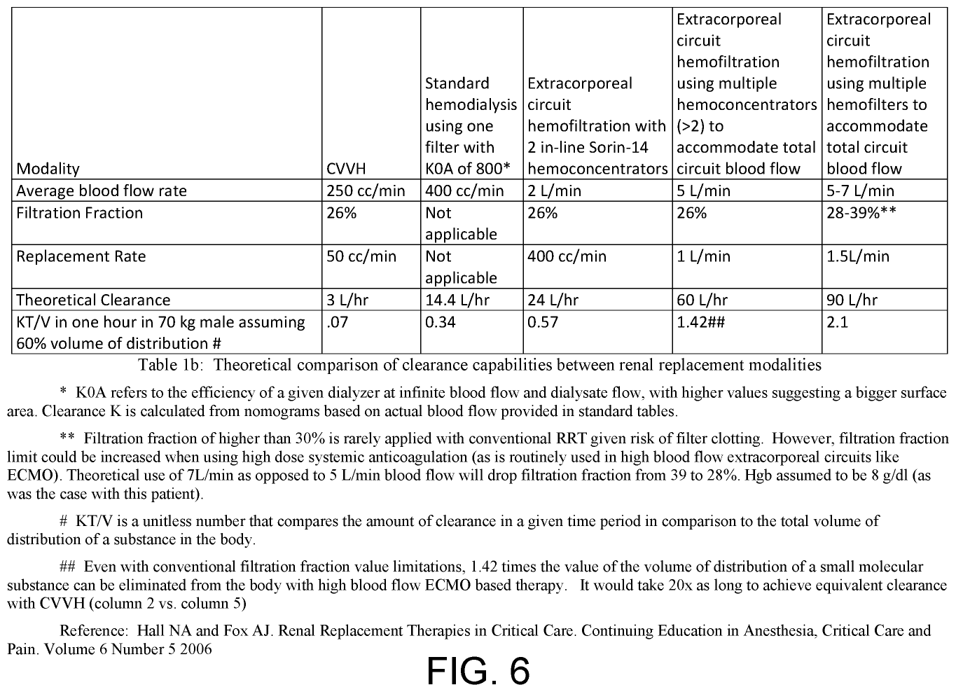

For the subsequent treatment (Treatment 2), a second Sorin-14 hemofilter was placed in-line, as shown in FIG. 4. Both hemofilters, now in parallel with one another, maintained blood flow throughput of ˜1 L/minute each as measured by flow meters. A similar procedure was followed to administer 5 L of replacement fluid along with matched effluent volume removal of 5 L via gravity drainage. However, during Treatment 2, effluent was removed simultaneously from both hemofilters via gravity drainage, which doubled the effluent removal rate. This process of replacing and removing 5 L of fluid was completed in 25 minutes, around 10% of the time required to achieve standard CRRT clearance goals of 20 cc/kg/hour. The patient remained hemodynamically stable (MAP 70-90) without any adverse effects and there was no net fluid removal. Traditional CRRT was subsequently initiated for further convective clearance and volume management. Basic metabolic panels were obtained pre and post both treatment sessions, the results of which are shown in Table la of FIG. 5. Effluent fluid was collected and sodium, potassium, and creatinine values were measured from both sessions, as shown in Table 1b of FIG. 6.

As exemplified in FIGS. 5 and 6, high-efficiency hemofiltration is easily able to rival hemodialysis with two filters in parallel, and clearance markedly increases at maximal blood flows. If system coagulation is used to prevent filter clotting, traditional filtration fraction limits of 20% can be exceeded and clearance enhanced even further (right-most column of FIG. 6).

Traditional calculations for clearance in CRRT using post-filter replacement fluid utilize the concept that K (clearance)=hemofiltration rate×S (the ratio of effluent solute concentration/blood solute concentration). Theoretically, S=1 for most small solutes like Cr given that they freely pass through membrane pores during convection. In the exemplary patient discussed here, as anticipated, the S value for Cr was nearly one. The above replacement rates (5 L/time of treatment) normalized to 1 hour yielded a theoretical K (clearance) value of 6 L/hour in Treatment 1. In Treatment 2, the K value was twice the value of Treatment 1 (12 L/hour), given that filter area doubled during the second treatment. Clearance can also be calculated as measured mass flux across the membrane (J)/average blood solute concentration (FIG. 5). With this approach, the Cr clearance for Treatment 1 was 8.3 L/hour, whereas in Treatment 2, it was 11.2 L/hour. Differences between the theoretical and calculated values may represent inherent lab variability when measuring small concentrations of Cr in effluent samples or drug interference with the Cr assay. The overall trend of both calculations, however, shows near doubling of clearance when two filters were used in parallel approximating the upper limit of what conventional CRRT is currently able to achieve. This was using only 1 L/minute of blood flow per hemofilter, whereas the ECMO circuit can actually flow ˜5-7 L/minute. This high blood flow could theoretically allow for even greater convective clearance (FIG. 6).

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

1. A device for processing blood flow in an extracorporeal circuit, comprising:
a high-flux hemofilter assembly, said high-flux hemofilter assembly further comprising:
one or more blood inlets in fluid communication with a patient blood drainage line;
one or more blood outlets in fluid communication with a patient blood return line; and
an effluent outlet;
wherein said high-flux hemofilter assembly is configured to provide solute clearance on blood supplied through said one or more blood inlets at a blood flow rate of 1-7 L/min, and wherein said solute clearance is effective to cause small solute clearance from said blood supplied through said one or more blood inlets at a rate of 24-90 L/hr.

2. The device for processing blood flow of claim 1, said high-flux hemofilter assembly further comprising a plurality of hemofilters arranged in parallel flow between said patient blood drainage line and said patient blood return line.

3. (canceled)

4. (canceled)

5. The device for processing blood flow of claim 1, said high-flux hemofilter assembly further comprising a single hemofilter arranged in series flow between said patient blood drainage line and said patient blood return line.

6. A system for processing blood flow in an extracorporeal circuit, comprising:
a high-flux hemofilter assembly, said high-flux hemofilter assembly further comprising:
one or more blood inlets in fluid communication with a patient blood drainage line;
one or more blood outlets in fluid communication with a patient blood return line; and
an effluent outlet;
wherein said high-flux hemofilter assembly is configured to provide solute clearance on blood supplied through said one or more blood inlets at a blood flow rate of 1-7 L/min, and wherein said solute clearance is effective to cause small solute clearance from said blood supplied through said one or more blood inlets at a rate of 24-90 L/hr; and
a fluid selected from the group consisting of a replacement fluid and a dialysate, wherein said fluid is in fluid communication with said extracorporeal circuit.

7. The system for processing blood flow of claim 6, said high-flux hemofilter assembly further comprising a plurality of hemofilters arranged in parallel flow between said patient blood drainage line and said patient blood return line.

8. (canceled)

9. The system for processing blood flow of claim 6, wherein each of said hemofilters in said plurality of hemofilters is configured to provide solute clearance on blood supplied through said one or more blood inlets at a blood flow rate of 4-6 L/min.

10. The system for processing blood flow of claim 6, said high-flux hemofilter assembly further comprising a single hemofilter arranged in series flow between said patient blood drainage line and said patient blood return line.

11-14. (canceled)

15. The system for processing blood flow of claim 6, further comprising a replacement fluid pump in fluid communication with said fluid and said extracorporeal circuit.

16. The system for processing blood flow of claim 15, wherein said replacement fluid pump is configured to provide replacement fluid to said extracorporeal circuit at a rate that matches a hemofiltration rate of said high-flux hemofilter assembly.

17. The system for processing blood flow of claim 6, further comprising an effluent pump in fluid communication with said effluent outlet of said high-flux hemofilter assembly and an effluent waste collection vessel.

18. (canceled)

19. The system for processing blood flow of claim 6, further comprising a centrifugal pump in fluid communication with said patient blood drainage line, wherein said centrifugal pump is configured to supply blood to said high-flux hemofilter assembly at a blood flow rate of 1-7 L/min.

20. A method for processing blood flow in an extracorporeal circuit, comprising the steps of:
providing a high-flux hemofilter assembly, said high-flux hemofilter assembly further comprising:
one or more blood inlets in fluid communication with a patient blood drainage line;
one or more blood outlets in fluid communication with a patient blood return line; and
an effluent outlet;
wherein said high-flux hemofilter assembly is configured to provide solute clearance on blood supplied through said one or more blood inlets at a blood flow rate of 1-7 L/min, and wherein said solute clearance is effective to cause small solute clearance from said blood supplied through said one or more blood inlets at a rate of 24-90 L/hr;
providing a fluid selected from the group consisting of a replacement fluid and a dialysate, wherein said fluid is in fluid communication with said extracorporeal circuit;
supplying blood from a patient to said high-flux hemofilter assembly at a blood flow rate of 1-7 L/min; and
operating said high-flux hemofilter assembly to perform solute clearance on said blood effective to cause small solute clearance from said blood at a rate of 24-90 L/hr.

21. The method for processing blood flow of claim 20, wherein said high-flux hemofilter assembly further comprises a plurality of hemofilters arranged in parallel flow between said patient blood drainage line and said patient blood return line.

22. (canceled)

23. The method for processing blood flow of claim 20, further comprising the step of operating said extracorporeal circuit to provide solute clearance on blood supplied through said one or more blood inlets at a blood flow rate of 4-6 L/min.

24. The method for processing blood flow of claim 20, wherein said high-flux hemofilter assembly further comprises a single hemofilter arranged in series flow between said patient blood drainage line and said patient blood return line.

25. (canceled)

26. The method for processing blood flow of claim 20, wherein said fluid includes urea at a concentration of 20-200 mg/dL.

27. (canceled)

28. The method for processing blood flow of claim 20, wherein said fluid further comprises one or more substances selected from the group consisting of:
phosphorous; iron; thiamine; riboflavin; vitamin B-6; vitamin B-12; niacin; folic acid;
vitamin C; and combinations of any of the foregoing.

29. The method for processing blood flow of claim 20, further comprising the step of supplying said fluid to said extracorporeal circuit at a rate that matches a hemofiltration rate of said high-flux hemofilter assembly.

30. The method for processing blood flow of claim 20, further comprising the step of withdrawing effluent from said high-flux hemofilter assembly through said effluent outlet at an effluent flow rate of up to 2 L/min.

31. The method for processing blood flow of claim 20, wherein said step of providing a fluid in fluid communication with said extracorporeal circuit further comprises running said fluid counter current to blood flow in said high-flux hemofilter assembly from a side of a hemofilter membrane to allow for diffusive clearance that is opposite a blood supply side of said hemofilter membrane.

32. (canceled)

33. (canceled)
